(19) United States
(12) Patent Application Publication
(10) Pub. No.: US 2004/0077615 A1
(43) Pub. Date: Apr. 22, 2004

(54) Novel formulations for administering therapeutic lipophilic molecules

(76) Inventors: Katherine D. Gordon, Winchester, MA (US); Robert J. Leonard, Wellesley, MA (US)

Correspondence Address: BROMBERG & SUNSTEIN LLP, 125 SUMMER STREET, BOSTON, MA, 02110-1618, US

(21) Appl. No.: 10/463,192

(22) Filed: Jun. 17, 2003

Related U.S. Application Data
(63) Continuation of application No. 09/932,821, filed on Aug. 17, 2001.
(60) Provisional application No. 60/226,106, filed on Aug. 17, 2000.

(51) Int. Cl. A61K 31/56

(52) U.S. Cl. 514/182

(57) ABSTRACT

Method and compositions are described for treating an acute medical condition in a subject with a lipophilic agent, where the lipophilic agent is formulated in an oil, the formulation optionally including benzyl alcohol. The formulation is suited for administering the lipophilic agent subcutaneously to achieve rapid uptake by the blood and thereafter sustained delivery.

### CROSS REFERENCE TO RELATED APPLICATIONS

This application is a continuation from U.S. patent application Ser. No. 09/932,821 filed Aug. 17, 2001 which in turn claims priority from provisional application 60/226,106 filed Aug. 17, 2000. These related applications are hereby incorporated herein by reference.

### TECHNICAL FIELD AND BACKGROUND ART

The present invention relates to the acute delivery of lipophilic agents by a subcutaneous route of delivery in the presence of an oil and optionally benzyl alcohol.

Oral delivery of active agents is commonly the preferred route of drug delivery in humans and animals as it is non-invasive and the drugs in a fixed dose are readily and conveniently administered. However, oral drug delivery has certain disadvantages which include poor bioavailability, the need to formulate and administer excess amounts of drugs beyond the dose that is therapeutically required and a relatively slow rate of delivery into the plasma from the stomach and gastrointestinal tract.

Intravenous drug delivery is the most rapid route of delivery into the blood of a patient. However, intravenous injection frequently requires a hospital setting with skilled personnel administering the agent. Moreover, intravenous injection causes discomfort to the patient. In addition, intravenous administration requires that the agent be formulated in an aqueous carrier. This requirement can limit the use of lipophilic agents for intravenous injection. This has been overcome in circumstances where the lipophilic agent is made available in a salt (conjugate) form which can dissolve in an aqueous formulation. However, the salt or conjugate must be convertible in vivo into the active agent. Alternatively, cyclodextrins, a group of cyclic saccharides can form inclusion complexes with poorly water-soluble compounds to increase their aqueous solubility. Other routes of delivery include intramuscular and transdermal routes. These methods of delivery are particularly suited for sustained and long term (chronic) administration of an active agent. Just as with intravenous administration of an agent, intramuscular administration is frequently uncomfortable for the patient.

There are a number of medical conditions for which it is necessary to administer an active agent rapidly. For example, after an ischemic event, time is of the essence as oxygen deprived tissues become rapidly damaged, exacerbated by oxidative damage during reperfusion. Other conditions that require acute and sustained delivery of agents include an allergic response to an allergen or response to other toxins that can quickly develop into anaphylactic shock or other symptoms. Epileptic seizures require rapid anticonvulsant intervention to protect the patient from damage to the central nervous system.

An example of one class of lipophilic molecule is the class of estrogens, which readily cross the blood brain barrier and protect animal and human subjects from neurodegenerative diseases and stroke (see for example, Hurn and Macrae 2000, Blood Flow and Metabolism, Vol. 20, pp. 631-652), These compounds are candidates for rapid delivery to target sites to treat acute episodes of disease in the brain. Formulations that provide rapid delivery of a lipophilic agent in an easy to use format would provide improved treatments for a variety of acute medical conditions.

### SUMMARY OF THE INVENTION

In a first embodiment of the invention there is provided a method for treating an acute medical condition with a lipophilic agent, that includes: providing the lipophilic agent in an oil formulation in the presence of benzyl alcohol, and administering the formulation subcutaneously (i) to provide a peak plasma concentration of the lipophilic agent within 4 hours after the subcutaneous administration; and (ii) to achieve sustained delivery.

According to the above, the lipophilic molecule may include a polycyclic phenolic compound exemplified by a steroid, more particularly an estrogen. The lipophilic molecule may further be a benzodiazapine, for example diazepam.

The oil may be one or more vegetable oils where the vegetable oil is selected from the group consisting of corn, sesame, cottonseed, soybean, poppy seed, castor, olive, canola, rapeseed, peanut, sunflower and mixtures thereof.

The medical condition may be an ischemic condition, a seizure or trauma for example: stroke, subarachnoid hemorrhage, cerebrovascular injury, vasospasm, head injury, myocardial infarction and angina, epilepsy or trauma.

In an embodiment of the invention, a dosage unit for subcutaneous administration may include a formulation of a non-estrogenic lipophilic molecule (for example having a molecular weight less than 1000D) dissolved in an oil packaged in a dosage unit for subcutaneous delivery. The lipophilic agent may be a polycyclic compound with a terminal phenol group. The lipophilic agent may be a benzodiazepine, including for example, diazepam.

In an embodiment of the invention, a dosage unit for subcutaneous administration is provided that includes a formulation of a lipophilic molecule dissolved in an oil and benzyl alcohol, packaged in a dosage unit for subcutaneous delivery.

The lipophilic agent can be a polycyclic compound with a terminal phenol group, for example, a steroid, an example of a steroid being estrogen. The lipophilic compound can be a benzodiazepine, for example diazepam.

In an embodiment of the invention, a method is provided of treating an acute medical condition with, a non-estrogenic lipophilic agent, that includes providing the non-estrogenic lipophilic agent in an oil formulation, and administering the formulation subcutaneously (i) to provide a peak plasma concentration of the lipophilic agent within 4 hours after the subcutaneous administration; and (ii) to achieve sustained delivery.

According to the above, the lipophilic agent may be polycyclic compound with a terminal phenol group or a benzodiazepine for example, diazepam.

### BRIEF DESCRIPTION OF THE DRAWINGS

The foregoing features of the invention will be more readily understood by reference to the following detailed description, taken with reference to the accompanying drawings, in which:

FIG. 1 shows the pharmacokinetic profile of diazepam following subcutaneous administration in an oil vehicle in healthy adult subjects compared to other delivery modalities. —⋄—, subcutaneous delivery (5 mg); —▪—, intravenous delivery (7.5 mg) —•—, rectal delivery (15 mg). Mean values are provided at each time point.

FIG. 2 shows the pharmacokinetic profile in rats of 17β-estradiol following subcutaneous administration in an oil vehicle in the absence (a) or presence (b) of 2% benzyl alcohol. Standard deviations of the mean are shown for each time point.

FIG. 3 shows the pharmacokinetic profile in rats of 17β-estradiol following subcutaneous administration in a vehicle of corn oil (a), sesame oil (b) and cotton seed oil (c). Standard deviations of the mean are shown for each time point.

### DETAILED DESCRIPTION OF SPECIFIC EMBODIMENTS

Definitions. As used in this description and the accompanying claims, the following terms shall have the meanings indicated, unless the context otherwise requires: “Lipophilic” compounds are those compounds that have greater solubility in oil than in aquous medium. Accordingly, lipophilic compounds may have an octanol: water partition coefficient (at room temperature, generally about 23° C.) of greater than 10:1, more preferably greater than 50:1, and even more preferably greater than 100:1

“Estrogen compounds” are any of the structures in the 11^{th} Edition of “Steroids” from Steraloids Inc. herein incorporated by reference. Included are the compounds itemized in the definition of estrogen compounds provided in U.S. Pat. No. 5,554,601, herein incorporated by reference.

“Benzodiazepines” are lipophilic agents that potentiate the activity of the gamma—aminobutyric acid (GABA) neurotransmitter in the central nervous system and enhance the opening of the chloride channel on neurons which in turn inhibit cellular excitation. Benzodiazepines are used to control seizures, multiple forms of panic attacks, anxiety, alcohol withdrawal, insomnia and for conscious sedation before medical procedures.

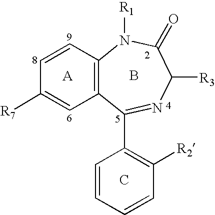

Benzodiazapine (1-Phenyl-1,4-benzodiazepin-2-one)

R₂ may be unsubstituted or contain halogen atoms

R₁ can be H, CH₃ or lower alkyl groups

The amide group can be replaced with an amidine group as in chlordiazepoxide

Amide can be replaced with heterocycle such as imidazole or trizole

The 3-position may be unsubstituted (R₃═H) or hydroxylated (R₃═OH).

Benzodiazepine products include N-1 substituted 3-unsubstituted benzodiazepines (diazepams) which further include diazepam, prazepam, flurazepam, halezepam, quazepam and flunitrazepam. Benzodiazepine products further include aminidino-N-oxide benzodiazepines (chlordiazepoxide); 3-carboxy-N-1-unsubstituted benzodiazepines (chlorazapate); N-1-substituted-3-hydroxy benzodiazepines (N-alkyl oxazepams); N-1-unsubstituted-3-hydroxy benzodiazepines (oxazepams) including oxazepam and lorazepam; imidazo-benzodiazepines including midazolam; and triazolo-benzodiazepines including alprazolam, triazolam and estazolam and enantiomeric forms. Included in benzodiazepines are the metabolic products of the above molecules. (also incorporated herein by reference is Jack DeRuiter, Principles of Drug Action, (Fall 2000), Vol. 2, on benzodiazepines).

“Polycyclic compounds with a terminal phenol group” include compound having a terminal phenolic ring and at least a second carbon ring. In addition to these required structures, the compound may have a number of R groups attached to any available site on the phenolic ring or elsewhere providing that the phenolic structure of the terminal ring is maintained. These R-groups may be selected from inorganic or organic atoms or molecules. Below, examples of a number of different types of R groups have been provided although the invention is not limited by these examples.

(a) The R group may include any inorganic R group including any of a halogen, an amide, a sulfate, a nitrate, fluoro, chloro, or bromo groups. Additionally, R groups selected from sodium, potassium and/or ammonium salts may be attached to the alpha or beta positions to replace hydrogen on any available carbon in the structure. The R-group may be organic or may include a mixture of organic molecules and ions. Organic R groups may include alkanes, alkenes or alkynes containing up to six carbons in a linear or branched array. For example, additional R group substituents may include methyl, ethyl, propyl, butyl, pentyl, hexyl, heptyl, dimethyl, isobutyl, isopentyl, tert-butyl, sec-butyl, isobutyl, methylpentyl, neopentyl, isohexyl, hexenyl, hexadiene, 1,3-hexadiene-5-yne, vinyl, allyl, isopropenyl, ethynyl, ethylidine, vinylidine, isopropylidene; methylene, sulfate, mercapto, methylthio, ethylthio, propylthio, methylsulfinyl, methylsulfonyl, thiohexanyl, thiobenyl, thiopenol, thiocyanato, sulfoethylamide, thionitrosyl, thiophosphoryl, p-toluenesulfonate, amino, imino, cyano, carbamoyl, acetamido, hydroxyamino, nitroso, nitro, cyanato, selecyanato, arccosine, pyridinium, hydrazide, semicarbazone, carboxymethylamide, oxime, hydrazone, sulfurtrimethylammonium, semicarbazone, o-carboxymethyloxime, aldehyde hemiacetate, methylether, ethylether, propylether, butylether, benzylether, methylcarbonate, carboxylate, acetate, chloroacetate, trimethylacetate, cyclopentylpropionate, propionate, phenylpropionate, carboxylic acid methylether, formate, benzoate, butyrate, caprylate, cinnamate, decylate, heptylate, enanthate, glucosiduronate, succinate, hemisuccinate, palmitate, nonanoate, stearate, tosylate, valerate, valproate, decanoate, hexahydrobenzoate, laurate, myristate, phthalate, hydroxyl, ethyleneketal, diethyleneketal, formate, chloroformate, formyl, dichloroacetate, keto, difluoroacetate, ethoxycarbonyl, trichloroformate, hydroxymethylene, epoxy, peroxy, dimethyl ketal, acetonide, cyclohexyl, benzyl, phenyl, diphenyl, benzylidene, and cyclopropyl groups. R groups may be attached to any of the constituent rings to form a pyridine, pyriazine, pyrimidine, or v-triazine. Additional R group substituents may include any of the six member or five member rings itemized in section b below.

(b) Any compound having in addition to the phenol A ring, a heterocyclic carbon ring which may be an aromatic or non-aromatic phenolic ring with any of the substitutions described in (a) above and further may be selected from for example, one or more of the following structures—phenanthrene, naphthalene, napthols, diphenyl, benzene, cyclohexane, 1,2-pyran, 1,4-Pyran, 1,2-pyrone, 1,4-pyrone, 1,2-dioxin, 1,3-dioxin (dihydro form), pyridine, pyridazine, pyrimidine, pyrazine, piperazine, s-triazine, as-triazine, v-triazine, 1,2,4-oxazine, 1,3,2-oxazine, 1,3,6-oxazine (pentoxazole), 1,2,6 oxazine, 1,4-oxazine, o-isoxazine, p-isoxazine, 1,2,5-oxathiazine, 1,2,6-oxathiazine, 1,4,2-oxadiazine, 1,3,5,2-oxadiazine, morpholine (tetrahydro-p-isoxazine), any of the six ringed structure listed above being a terminal group in the compound. Additionally, any of the above carbon ring structure may be linked directly or via a linkage group to any further heterocyclic aromatic or non aromatic carbon ring including: furan; thiophene (thiofuran); pyrrole (azole); isopyrrole (isoazole); 3-isopyrrole (isoazole); pyrazole (1,2-daizole); 2-isoimidazole (1,3-isodiazole); 1,2,3-triazle; 1,2,4 triazole; 1,2-diothiole; 1,2,3-oxathiole, isoxazole (furo(a) monozole); oxazole (furo(b) monazole); thiazole; isothiazole; 1,2,3-oxadiazole; 1,2,4-oxadiazole, 1,2,5-oxadiazole, 1,3,5 oxadiazole, 1,2,3,4-oxatiazole; 1,2,3,5-oxatriazole; 1,2,3-dioxazole; 1,2,4-dioxazole; 1,3,2-dioxazole; 1,3,4-dioxazole; 1,2,5-oxathiazole; 1,3-oxathiole, cyclopentane. These compounds in turn may have associated R groups selected from section (a) or section (b) above that are substituted on the carbon ring at any of the available sites.

(c) Any compound including those listed above, that may form a cyclopentanophen(a)anthrene ring compound and which, for example may be selected from the group consisting of 1,3,5 (10), 6,8-estrapentaene, 1,3,5 (10), 6,8,11-estrapentaene, 1,3,5 (10) 6,8,15-estrapentaene, 1,3,5 (10), 6,-estratetraene, 1,3,5 (10), 7-estratetraene, 1,3,5 (10)8-estratetraene, 1,3,5 (10)16-estratetraene, 1,3,5 (10)15-estratetraene, 1,3,5 (10)-estratriene, 1,3,5 (10) 15-estratriene.

(d) Any compound including precursors or derivatives selected from raloxifen, tamoxifen, androgenic compounds, and their salts where an intact phenol ring is present with a hydroxyl group present on carbons 1, 2, 3 and 4 of the terminal phenol ring.

(e) Any compound in the form of a prodrug, that may be metabolized to form an active polycyclic phenolic compound having neuroprotective activity.

An “acute” medical condition occurs when an adverse physiological event happens over minutes or hours or even several days resulting in potential long lasting damage or causing extreme discomfort to the patient during the episode if untreated.

“Subcutaneous” is below the skin usually in the connective tissue underlying the dermis and above the facia of the muscle tissue.

“Dosage unit” is a term of art for administering an effective dosage for treating a patient.

“Subject” refers to a human or animal.

We demonstrate for the first time, that an oil formulation for lipophilic drugs can produce a rapid uptake of the drug by subcutaneous administration with sustained delivery in a biphasic kinetic function. In addition, we have shown that the addition of benzyl alcohol which acts as a preservative, solubilizer and stabilizer, can increase the reproducibility of the pharmacokinetic profile.

According to embodiments of the invention, the oil can be of the type which generally is liquid at body temperatures, eg., about 37° C. including but not limited to: corn oil, sesame oil, cotton oil, soybean oil, poppy seed oil, castor oil, sesame oil, canola oil, olive oil, peanut oil, rapeseed oil and mixtures thereof.

Examples of types of lipophilic molecules for treating acute medical conditions include lipophilic benzodiazepines, antidepressants, antioxidants (eg., carotenoids, b-carotene, alpha-carotene, lycopene), angiotensin-converting enzyme inhibitors (eg., fosinopril, lisinopril), calcium channel antagonists (eg., bepridil), immunosuppressants (eg., cyclosporin A), anesthetics, antibiotics (eg., sparfloxacin, moxifloxacin, norfloxacin), anti-cancer compounds (eg., idarubicin, iododoxorubicin, 5-fluorouracil, etoposide, taxol), anti-fungal compounds, non-steroidal anti-inflammatory drugs (NSAIDS) (eg., indomethacin), anti-ulcer compounds (eg., omeprazole), beta-blockers (eg., metoprolol, propranolol), cholinesterase inhibitors (eg., physostigmine), anticoagulants (eg., heparin), bronchodilators, hypolipemics (eg., fenofibrate, clofibrate), steroids (estrogen compounds, polycyclic phenolic compounds), opiates, antiemetics (eg., serotonin blockers), and opidids (fentanyl).

The dosage of active drug within the formulation may be within the normal range while concentrations at the maximum soluble concentrations may be used. In the case of steroids, a concentration of 10 mg/ml or higher is desirable. Lower concentrations, such as 1-10 mg/ml or lower, can also be used. For benzodiazepines, concentrations of 1-10 mg/ml or lower can be used resulting in an administered dose of 6 mg or less per dose. The maximum soluble concentration can be easily determined empirically by visual or biochemical assessment as the formulation is produced.

The drug delivery compositions may contain in addition to the above, more than one solubilizer, stabilizer and/or preservative, more than one therapeutic molecule, and one or more types of oils. The compositions may also contain an antioxidant such as a-tocopherol or ascorbate and/or an antimicrobial compound. The drug delivery compositions may be stored at various temperatures preferably below 40° C.

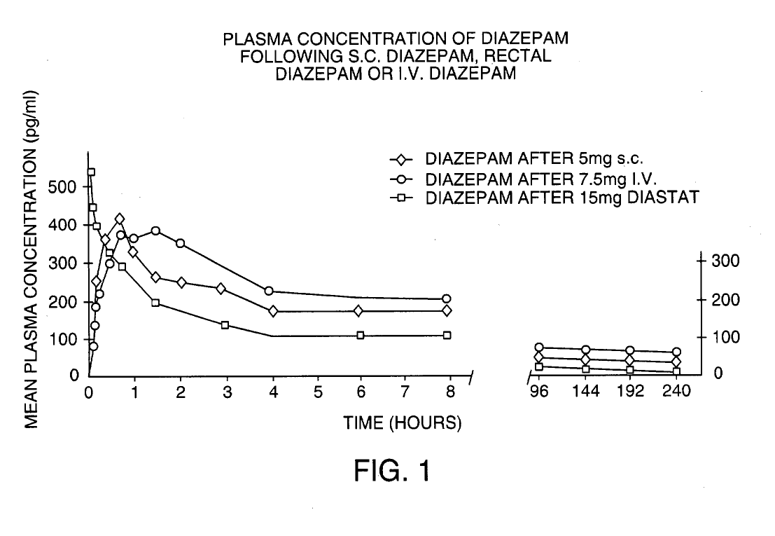

The novel formulation is well suited for delivery of therapeutic doses of a drug to a human or animal by subcutaneous injection in situations characterized by the need to attain peak concentrations of drug in the blood of the subject. The drug delivery vehicle can deliver an effective dose of the drug via one injection although multiple injections may also be performed. Maximum blood levels may be achieved within a four-hour period, for example within a 1 hour period. After about 4 hours, blood concentration of the drug is reduced but the drug remains in circulation for 8 hours or more (see FIGS. 1-3). In summary, the drug delivery formulation described here has the advantages of convenient rapid uptake into the bloodstream when applied in a single subcutaneous injection and further provides sustained delivery kinetics.

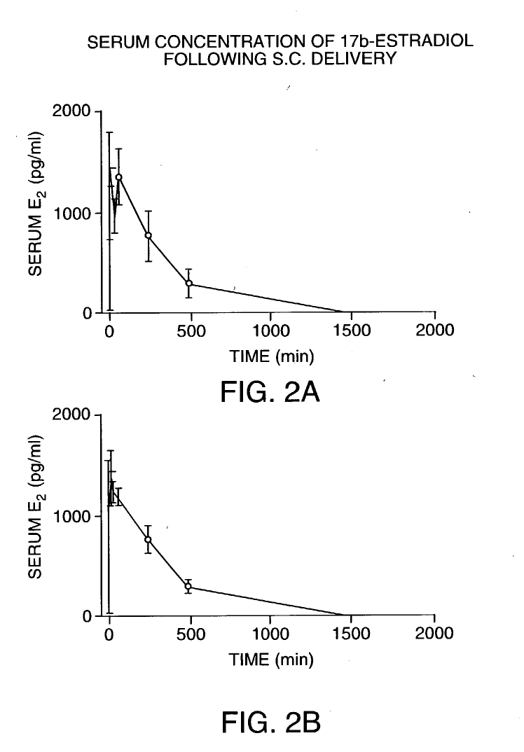

We have provided a general purpose formulation for delivery of lipophilic molecules useful for treating acute medical conditions in humans. In addition, we have identified the novel use of a molecule that is at the same time a solubilizer, a stabilizer and a preservative for improving the rate at which agents can be delivered into the plasma subcutaneously while preserving sustained delivery to the blood. In the examples, this molecule is exemplified by benzyl alcohol. (Handbook of Food, Drug and Cosmetic Excipients; CRC Press. herein incorporated by reference). As demonstrated in FIG. 2, the presence of benzyl alcohol reduces the fluctuation in uptake between different subjects subjected to a lipophilic agent in oil by a subcutaneous route of administration. The presence of benzyl alcohol provides a more consistent pharmacokinetic profile then is observed in its absence. This is a useful improvement because it permits the physician to recommend with a high degree of certainty, a dosage that will have the predicted pharmacokinetic profile for a patient.

The drug delivery formulation described above provides an improvement for treating diseases in which rapid achievement of high levels of drug in the blood are desirable; eg., acute diseases or conditions.

Examples of acute medical conditions in which rapid uptake of drug in the blood is desirable and for which the present embodiments directed to subcutaneous delivery can be especially useful include: cerebral and cardiovascular ischemia, particularly strokes, transient ischemic attacks, coronary vasospasms, cardiovascular events (including MI) in high risk individuals, atherosclerosis, neurodegenerative disorders, heart attacks, acute angina), asthma, hypertension, congestive heart failure (including myocardial infaracts), toxic effects of poisons including acute iron toxicity and snake bites, nephropathy (with or without diabetes mellitus), , cancer, depression, psychosis, anesthesia, analgesia, ulcer, hypertension, pain, urinary incontinence, coagulopathies, trauma such as caused by accidental damage including gun shot wounds, allergic response, transplantation, prevention of immune rejection and status epilepticus.

Subcutaneous administration of lipophilic agents is especially useful in acute situations when it is impossible or impractical to deliver a compound intraveneously for example status epilepticus. In status epilepticus, (SE) it is necessary to abort the seizures and treat the underlying inciting condition as rapidly as possible. Progressive neuronal damage occurs if convulsive SE persists for more than 30 minutes, with neurological, epileptic and cognitive sequelae. Continuous intravenous administration of specific antiepileptic drugs (eg., diazepam, lorazepam, midazolam or combined lorazepam and phenytoin) is traditionally the treatment of choice although the patient generally has to be hospitalized and trained personnel are required to deliver the agent. A single subcutaneous injection provides uptake in to the circulation that is rapid and more convenient than intravenous administration. Subcutaneous injection does not require skilled nurses to administer the drug. A dosage unit may optionally be administered from a prepackaged device that punctures the skin to the correct extent and releases the dosage. Moreover, subcutaneous administration is the cause of less discomfort to the subject than is normal with intramuscular and intravenous injections as well as with rectal suppositories.

Cerebral or cardiac ischemia is generally caused by a blockage of blood flow to the relevant tissue/organ followed by reperfusion. Reperfusion causes the formation of oxygen-derived free radicals and increases lipid peroxidation, resulting in tissue injury. When the blood supply to the brain or heart is blocked, a myocardial infarct (heart attack) or cerebral infarct (stroke) results and the deprived tissue dies with the result of permanent damage to the tissue. If the blood supply can be re-established within hours after infarction, the brain or heart tissue can remain viable and permanent damage can be reduced. This can be accomplished by surgical as well as pharmacological (thrombolysis) procedures and these processes lead to reperfusion. Reperfusion is now widely and successfully applied and it has been claimed that fatalities due to myocardial infarction or stroke can be reduced by 20-30%. However, reperfusion also poses problems. Oxygen-deprived (ischemic) tissue finds itself in an abnormal state and is vulnerable when suddenly exposed to oxygen-rich blood. This has been termed the “oxygen paradox” and leads to reperfusion damage in the form of cell death. Tissues subjected to transient ischemia or reperfusion in various disease states, or by their medical treatment, are those of heart, lung, kidney, pancreas and brain. Transient ischemia is one of the causative factors that lead to angina pectoris. In each of the aforementioned disease states, delivery of a drug that can protect against ischemic damage needs to occur as promptly as possible. The longer the process to provide biologically active drug to the patient, the more sustained the ischemic damage. Even death may ensue unless a rapid acting drug is delivered promptly. Thus, time is of the essence. The rapid-acting subcutaneous formulation described herein provides a means to deliver lipophilic therapeutics to treat these and other diseases and conditions resulting from ischemia.

Examples of therapeutics for treating ischemia include lipophilic polycyclic phenolic compounds exemplified by steroids, which can be delivered according to present embodiments of the invention for treating cerebral and cardiac ischemia. These types of compounds, which include estrogen compounds, protect neurons and other tissue types from damage, including damage caused by ischemia and oxygen radicals. (U.S. Pat. Nos. 5,554,601, 5,859,016, 5,843,934, 5,877,169, 5,972,923, 6,197,833 all incorporated by reference).

Lipophilic molecule such as estrogen in its native form when formulated with an oil plus benzyl alcohol and delivered by subcutaneous injection has here been shown to vary less in pharmacokinetic profile in different subjects then in the absence of the benzyl alcohol. Subcutaneous delivery of the formulation under the skin can be performed to maximize the surface area of the drug reservoir in situ, such that much of the total dose is immediately in contact with the local vasculature and rapid uptake is achieved. In this way, subcutaneous bolus injection is superior to an intramuscular depot preparation which is commonly used to administer oily suspensions.

The novel formulation has the advantages of convenient mode of administration of lipophilic molecules, rapid uptake into the bloodstream, sustained delivery of the drug in the bloodstream, and consistent interpersonal pharmacokinetics in the presence of benzyl alcohol.

### EXAMPLES

### Example 1

### Improved Pharmacokinetics for Benzodiazepine Administered Subcutaneously

Pharmacokinetics of diazepam administered by three different routes of administration were compared. These were (a) a rectal route, (b) an intravenous route, and (c) a subcutaneous injection.

Diazepam in the form of Diastat® (Elan) in a rectal suppository formulation was supplied at a concentration of 5 mg/ml in propylene glycol, ethanol, hydroxymethylcellulose and sodium benzoate (Physician's Desk Reference (PDR) ed. 2000, p 1012). Diazepam in an injectable format (Valium®, Roche) was supplied in a dosage unit containing 5 mg/ml of diazepam in polyethylene glycol, ethanol, sodium benzoate and benzyl alcohol (PDR ed. 2000, pp. 2677). We prepare diazepam for subcutaneous delivery as follows: Diazepam is dissolved in a small amount (about 1 ml) of dimethylsulfoxide (the “initial solvent source”) as a paste. Once dissolved, the sample is mixed with sesame oil, (NF grade (Penta)). The DMSO layer is separated from the oil layer and allowed to evaporate while the mixture is standing at room temperature within 10 hours to give a final concentration of 10 mg/ml. Although we have used DMSO here, other solvents such as ethanol or chloroform can also be effective. Also, although we have used sesame oil here, other types of oil are not precluded. The sublimation of the initial solvent can be performed at room temperature as is performed here. However, the sublimation can be also accomplished at higher temperatures optionally in a vacuum oven for shorter periods of time. Also, in this particular study, the final composition does not include benzyl alcohol or any other stabilizers or preservatives, although these can be added for improving the reproducibility of the pharmacokinetic profile.

Five healthy human volunteers (two female and three male) were recruited for testing each mode of delivery in the drug study. They were each given a single dose of the diazepam by rectal (15 mg), vascular injection (7.5 mg) or by subcutaneous route (5 mg). Samples of blood were taken over the next 8 hours and diazepam levels were measured in the plasma (see FIG. 1). As is apparent from this figure for subcutaneous delivery, levels in blood rise more quickly than via rectal delivery, reaching a peak concentration in less then one hour, and then falling off gradually, providing for rapid uptake kinetics as well as sustained delivery.

### Example 2

### Pharmacokinetic Measurements of Lipophilic Molecule Uptake by Plasma in Rats after Subcutaneous Administration in the Absence and Presence of Benzyl Alcohol.

To test the kinetics of uptake of an estrogen compound in an oil vehicle with benzyl alcohol, male Sprague-Dawley rats (Taconic) were given 17β-estradiol in a subcutaneous bolus injection, and drug levels in the blood were determined over a 24 hour period. The drug was dissolved in corn oil at 100 μg/ml and the final dosage delivered was 100 μg/kg. Blood samples were drawn at 30 minutes prior to drug administration, 15, 30 and 60 minutes after drug administration, as well as 4, 8 and 24 hours after drug administration. Venous blood was collected into heparinized tubes, centrifuged and the plasma was collected and frozen. Levels of 17β-estradiol were determined using a commercially supplied radioimmunoassay kit.

As shown in FIG. 2, there was a significant, very rapid uptake of the 17β-estradiol into the bloodstream, peaking within the 30 minute time point. By 24 hours, 17β-estradiol blood levels had fallen off to near zero. In panel (a), estradiol was formulated in oil and no benzyl alcohol was added. In panel (b) estradiol was formulated in oil and 2% benzyl alcohol. Comparisons of FIG. 2a to FIG. 2b demonstrates that the addition of the benzyl alcohol component results in the blood level of estradiol to form the tightest collection of data points at all time points.

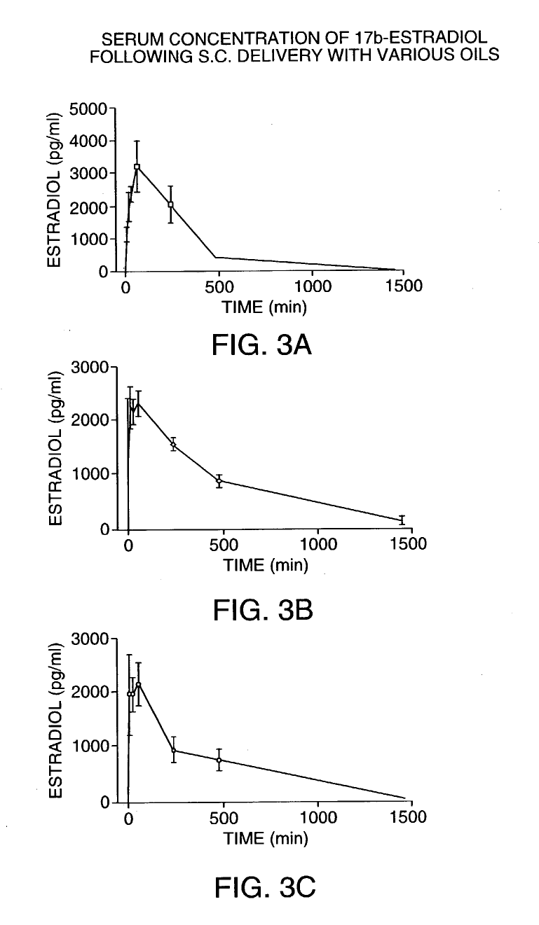

FIG. 3 which compares the kinetics obtained using formulations with 17β-estradiol, 2% benzyl alcohol and corn oil (panel A), sesame oil (panel B) or cotton seed oil (panel C) shows comparable results with these three oils.

Those skilled in the art will recognize or be able to ascertain using no more than routine experimentation, many equivalents to the specific embodiments of the invention described herein. Such equivalents are intended to be encompassed by the following claims.

What is claimed is:

1. A method for treating an acute medical condition in a subject with a polycyclic phenolic compound, comprising: providing the polycyclic phenolic compound in an oil formulation in the presence of benzyl alcohol, and administering the formulation subcutaneously to achieve sustained delivery.

2. The method according to claim 1, wherein the polycyclic phenolic compound is a steroid.

3. The method according to claim 2, wherein the steroid is an estrogen compound.

4. The method according to claim 3, wherein the estrogen is 17-beta-estradiol.

5. The method according to claim 1, wherein the oil is one or more vegetable oils.

6. The method according to claim 5, wherein the vegetable oil is selected from the group consisting of corn, sesame, cottonseed, soybean, poppy seed, castor, olive, canola, rapeseed, peanut, sunflower and mixtures thereof.

7. A method according to claim 1, wherein the medical condition is an ischemic condition or trauma.

8. A method according to claim 7, wherein the ischemic condition or trauma is selected from: a stroke, subarachnoid hemorrhage, cerebrovascular injury, vasospasm, head injury, myocardial infarction and angina.

9. A dosage unit for subcutaneous administration, comprising:
a formulation of a polycyclic phenolic compound in an oil formulation in the presence of benzyl alcohol, packaged in a dosage unit for subcutaneous delivery.

10. A dosage unit according to claim 9, wherein the polycyclic phenolic compound is a steroid.

11. A dosage unit according to claim 10, wherein the steroid is an estrogen compound.

12. A method of treating an acute medical condition in a subject with an estrogen compound, comprising: providing the estrogen compound in an oil formulation in the presence of benzyl alcohol, and administering the formulation subcutaneously to achieve sustained delivery.

13. A method according to claim 12, wherein the estrogen compound is 17-beta-estradiol.
